(19) United States
(12) Patent Application Publication
(10) Pub. No.: US 2005/0186235 A1
(43) Pub. Date: Aug. 25, 2005

(54) Compact powder cosmetic compositions with a solid fatty phase

(76) Inventors: Guenaelle Martin, Paris (FR); Agnes Themens, Bourg la Reine (FR); Maitena Leuridan, Paris (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/043,260

(22) Filed: Jan. 27, 2005

Related U.S. Application Data
(60) Provisional application No. 60/541,366, filed on Feb. 4, 2004.

(30) Foreign Application Priority Data
Jan. 27, 2004 (FR) 04 50148

(51) Int. Cl. A61K 7/00

(52) U.S. Cl. 424/401

(57) ABSTRACT

The present disclosure provides compact powder form cosmetic compositions for making up and/or caring for the skin, including the face, comprising a pulverulent phase and a fatty phase comprising at least one solid fatty phase, wherein the solid fatty phase is present in an amount such that the compact powder has a cohesion corresponding to a loss of mass, evaluated by the drop test, of less than 12% by weight, and the pulverulent phase is such that when it is used in a proportion of 91.2% by weight as a mixture with 8.8% of a reference liquid binder described in Example 1 and when the whole is compacted at a pressure of 2×10⁷ Pa, it results in a compact whose cohesion corresponds to a loss of mass, evaluated by the drop test, of greater than 12% by weight and/or a compact that does not erode homogeneously.

This non-provisional application claims the benefit of French Application No. 04/50148, filed Jan. 27, 2004, and U.S. Provisional Application No. 60/541,366, filed Feb. 4, 2004, both hereby incorporated by reference.

The present disclosure provides cosmetic compositions for making up and/or caring for the skin, such as the face.

The compositions disclosed herein may be used in makeup products, for example, for the face, and may have non-therapeutic care and/or treatment properties. They may be “matting” cosmetic compositions, i.e., compositions intended to reduce the sheen of greasy skin and/or to improve the long-term staying power of makeup, i.e., to prevent visual degradation in the course of the day.

Many makeup compositions of this type are in the form of compact powders generally comprising a fatty phase, conventionally known as a “binder,” and a pulverulent phase comprising pigments and/or fillers. The main function of the binder is to ensure sufficient cohesion of the final composition, i.e., to prevent its fragmentation, which may be caused by impacts, and to give it a good capacity to be taken up.

For these properties to be obtained, the other components of the cosmetic compositions, including the fillers they contain, also should be suitable for placing in a compact powder form.

However, certain fillers lack the ability to be compacted. These are referred to as “incompactable” fillers. Consequently, their presence in a cosmetic composition intended to be formulated in compact powder form may significantly affect the cohesion of the resulting powder, or even, when they are in large amount, make it impossible to obtain a corresponding compact powder using a mechanical press.

Low-density fillers may also raise a problem for the formulation of compact powders having satisfactory cohesion and/or cake-erosion characteristics. Thus, when such a filler is present in an excessive content, generally of greater than or equal to 5% by weight relative to the total weight of a composition intended to be compacted, it cannot be compacted satisfactorily with a standard binder such as isocetyl stearate or with the silicone binder described in patent application WO 93/17660, used at low content, for example, from about 3% to 5% by weight. However, increasing the binder content to improve the compacting, for example to a scale of 10% by weight of binder in the composition, generally proves to be detrimental to the surface appearance, which is then too greasy. Furthermore, the high cohesion thus obtained makes it difficult to erode the composition using an applicator such as a powder puff, brush, and even a finger.

Another alternative is disclosed in EP 0 717 979. This alternative comprises preparing “compact” powders from about 30% by weight of “incompactable” fillers, about 5% by weight of liquid binder and more than 50% of water, via a freeze-drying technique. However, this freeze-drying process can be a laborious technique to implement industrially, for instance, since it requires freezing of the intermediate composition.

There is thus a need for compact powder cosmetic compositions, which, although comprising large amounts of incompactable fillers and/or low-density fillers, nevertheless can have satisfactory cohesion and/or erosion properties, and are moreover not restricting in terms of industrial preparation.

The present inventors have found that it is possible to overcome the abovementioned drawbacks, by combining incompactable fillers and/or low-density fillers with a binder fatty phase having a specific composition.

In one aspect, the present disclosure provides cosmetic compositions in compact powder form for making up and/or caring for the skin, such as the face, comprising a pulverulent phase and a fatty phase comprising at least one solid fatty phase, wherein:
the at least one solid fatty phase is present in an amount such that the compact powder has a cohesion corresponding to a loss of mass, evaluated by the drop test, of less than 12% by weight, and
the pulverulent phase being such that when it is used in a proportion of 91.2% by weight as a mixture with 8.8% by weight of a reference liquid binder as defined in Example 1 and when the whole is compacted at a pressure of 2×10⁷ Pa (200 bar), it results in a compact whose cohesion corresponds to a loss of mass, evaluated by the drop test, of greater than 12% by weight and/or a compact that does not erode homogeneously.

In another aspect, the present disclosure provides cosmetic compositions in compact powder form for making up and/or caring for the skin, such as the face, comprising:
a pulverulent phase comprising at least one incompactable filler in an amount of greater than or equal to 5% by weight relative to the total weight of the composition, and
at least one fatty phase comprising at least one solid fatty phase, the solid fatty phase being present in an amount such that the weight ratio of the solid fatty phase to the at least one incompactable filler is greater than or equal to 1.

In another aspect, the present disclosure provides cosmetic compositions in compact powder form for making up and/or caring for the skin, such as the face, comprising:
a pulverulent phase comprising at least one low-density filler having an untamped density ranging from 0.01 to 0.50 g/cm³ and which is present in an amount greater than or equal to 5% by weight, relative to the total weight of the composition, and
at least one fatty phase comprising at least one solid fatty phase, the at least one solid fatty phase being present in an amount such that the weight ratio of the at least one solid fatty phase to the at least one low-density filler is greater than or equal to 1.

In yet another aspect, the present disclosure provides cosmetic compositions in compact powder form for making up and/or caring for the skin, such as the face, comprising:
a pulverulent phase,
a solid fatty phase, and
from 4% to 15% by weight relative to the total weight of the composition of a liquid fatty phase.

In still another aspect, the present disclosure provides cosmetic compositions in compact powder form for making up and/or caring for the skin, such as the face, comprising:
a pulverulent phase,
from 12% to 35% by weight relative to the total weight of the composition of a fatty phase, the fatty phase comprising at least from 8% to 25% by weight relative to the total weight of the composition of a solid fatty phase.

According to some embodiments, the compositions disclosed herein do not comprise 10% by weight of carnauba wax, 6% by weight of magnesium stearate and 5% by weight of amorphous magnesium and/or aluminium silicate, relative to the total weight of the composition.

In another aspect, the present disclosure provides the use of either a pulverulent phase as described above, or of at least one filler chosen from incompactable fillers and low density fillers in an amount of greater than or equal to 5% by weight relative to the total weight of the composition, in combination with a fatty phase comprising at least one solid fatty phase, for example, as described above for the preparation of a compact powder cosmetic compositions with satisfactory cohesion and erosion properties.

According to still another aspect, the present disclosure provides processes for making up and/or caring for the skin, comprising applying to the skin at least one composition decribed herein.

The compositions disclosed herein can have satisfactory cohesion and erosion properties, i.e., satisfactory stability on storage and do not change or undergo little change when subjected to impacts. The compositions also have a good capacity for take-up and application.

Cohesion and Erosion Measurements

Cohesion and erosion may be measured according to the protocols described below.

The pulverulent phase requires preformulation with a binder known as a reference liquid binder. The composition of the reference liquid binder, which is also given in Example 1 below, is as follows:
40.91 g of 2-ethylhexyl 4-para-methoxycinnamate (Parsol MC® from the company Roche Vitamins),
25 g of isocetyl stearate,
25 g of polydimethylsiloxane 10 cst (Fluid DC 200® 10 cst from Dow Corning), and
9.09 g of the mixture of preserving agents Phenonipe from the company Nipa (Clariant), described below.

The mixture of preserving agents Phenonip® contains (weight %):
70% of 2-phenoxyethanol,
17.4% of methyl p-hydroxybenzoate,
4.2% of ethyl p-hydroxybenzoate,
4.2% of butyl p-hydroxybenzoate,
2.1% of propyl p-hydroxybenzoate, and
2.1% of isobutyl p-hydroxybenzoate.

The mixture of reference liquid binder (8.8% by weight) and of the pulverulent phase (91.2% by weight) is placed in a 57×39×05 mm rectangular aluminium crucible.

The mixture (10.5 g) is poured into the crucible and a pressure of 2×10⁷ Pa (200 bar) is then applied.

It is estimated that the product is not eroded homogeneously when, once the surface of the product, which is compact in the crucible, is rubbed with the fingers or with a dry sponge, for example a rectangular sponge such as the sponge sold under the name Yukilon Standard® by the company Yukigaya, it crumbles, comes away in lumps and/or wears away by hollowing. The erosion of the product is then considered as being irregular and non-homogeneous over the entire surface.

The drop test protocol consists of subjecting the compact, which is in a proportion of 10.5 g in a crucible as defined above, to 10 standardized drops over a height of 20 cm onto a sandstone floor tile 30 mm thick; the base of the dish is oriented towards the receiving surface of the sandstone floor tile. The loss of mass of product is then measured, after which the percentage loss of mass of powder is determined.

When the compositions have good cohesion properties, they may have a loss of mass in the drop test of less than 12% by weight, for example, less than 10% by weight, or even less than 8% by weight.

As previously specified, the compositions are provided in the form of compact powders. This compacted feature is obtained by subjecting to a compression the mixture of the particulate phase and the associated fatty binder.

The compositions in the form of a compact powder may be prepared by mixing the whole components of the particulate phase (fillers and pigments) and then adding under agitation to this mixture the components constituting the fatty phase. The mixture is then ground, sieved, and then poured into an assay crucible and compacted. This compacting is generally realized using a press applying a pressure from 50 to 250 bars to obtain the compact powder.

Consequently, the compositions are different from the termed poured particulate compositions, which are just prepared by mixing a fatty phase in the molded state with a particulate phase.

Since the compositions of the present disclosure are provided in the form of a compact powder, they have a better chance, compared to poured-type compositions, of being disintegrated in the form of “free” solid particles.

Thus, one can, after the unpacking of a compact composition as presently disclosed, recuperate its particulate phase, which is not possible with poured compositions.

The unpacking can be done according to the following process: the compacted product is broken using a spatula over a sieve (250 p) and then the obtained heaps of powder are sieved. Thus, the particulate phase of the compact powder is recovered in the form of “free” solid particles.

Pulverulent Phase

The term “pulverulent phase,” as used herein, includes any solid compound and mixture of solid compounds whose melting point is higher than 250° C. and may even be much higher.

The pulverulent phase generally comprises at least one filler and/or at least one pulverulent dyestuff.

The at least one filler may be chosen from incompactable fillers, low-density fillers, additional fillers, and mixtures thereof.

The term “additional filler,” as used herein, means any filler other than the incompactable fillers and/or the low-density fillers described herein.

The at least one pulverulent dyestuff may be chosen from pigments and nacres usually used in cosmetic and/or dermatological compositions.

In some embodiments, the at least one filler is chosen from hydrophobic- treated and hydrophobic-coated powders.

The term “hydrophobic-treated and hydrophobic-coated powders,” as used herein, means any powder surface-treated with a hydrophobic agent, for example, as described below.

The hydrophobic agent may be chosen from, for example, silicones, including methicones, dimethicones, and perfluoroalkylsilanes; fatty acids, for example stearic acid; metal soaps, for example aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, amino acids; N-acylamino acids or salts thereof; lecithin, isopropyl triisostearyl titanate, and mixtures thereof.

The N-acylamino acids may comprise an acyl group having from 8 to 22 carbon atoms, for example a 2-ethylhexanoyl, caproyl, lauroyl, myristoyl, palmitoyl, stearoyl, or cocoyl group. The salts of these compounds may be, for example, the aluminium, magnesium, calcium, zirconium, zinc, sodium, or potassium salts. The amino acid may be, for example, lysine, glutamic acid or alanine.

The term “alkyl” as used above, denotes an alkyl group having from 1 to 30 carbon atoms, for example, from 5 to 16 carbon atoms.

Pigments and hydrophobic-treated fillers are described, for instance, in patent application EP 1,086,683 A.

The pulverulent phase of the compositions may comprise a hydrophobic- treated powder or mixture of powders in an amount of greater than or equal to 50% by weight, for example, greater than or equal to 60% by weight, or even greater than or equal to 70% by weight, relative to the total weight of the pulverulent phase.

As described above, the compositions disclosed herein may comprise at least one incompactable filler and/or at least one low-density filler.

Incompactable Fillers

In some embodiments, the compositions comprise at least one incompactable filler in an amount of greater than or equal to 5% by weight, relative to the total weight of the composition.

The term “incompactable filler,” as used herein, means a powder which, at and above a certain amount that will depend on the material under consideration, for example, from an amount of greater than or equal to 5% by weight relative to the total weight of the composition, does not have good compactability using a manual press, for example, under a pressure of 2×10⁷ Pa (200 bar) and/or is detrimental to the production of products with good impact strength, for example, having a loss of mass of greater than 12% in the drop test, and/or that does not make it possible to obtain a compact product that can be eroded homogeneously.

The at least one incompactable filler may be organic or mineral, and in spherical or lamellar form.

Incompactable spherical fillers that may be used include:
silica microspheres, for example of open porosity, such as hollow silica microspheres, including the products “Silica Beads SP 700/HA®” and “Silica Beads SB 700®” from the company Maprecos, and “Sunspheres H-33®” and “Sunspheres H-51®” from the company Asahi Glass; these microspheres may, where appropriate, be impregnated with a cosmetic active agent,
microporous polymer microspheres, which have a structure similar to that of a sponge; they generally have a specific surface area of at least 0.5 m² μg, for example, of at least 1 m²/g, the specific surface area having no upper limit other than that resulting from the practical possibility of making microspheres of very high porosity: the specific surface area may be, for example, up to 1000 m²/g or even more. Examples of these microspheres include acrylic polymer microspheres, such as those made of crosslinked acrylate copolymer “Polytrap 6603 Adsorber®” from the company RP Scherrer, and those made of polymethyl methacrylate “Micropearl M 100®” from the company SEPPIC,
polyurethane powder, such as the powdered copolymer of hexamethylene diisocyanate and of trimethylolhexyl lactone sold under the name “Plastic Powder D-400®” by the company Toshiki,
polymer microcapsules having only one closed cavity and forming a reservoir, which may contain a liquid, for example, a cosmetic active agent; these polymer microcapsules are prepared via known processes, such as those described in patents U.S. Pat. No. 3,615,972 and EP 56,219 A. They may be made, for example, of polymers or copolymers of ethylenically unsaturated acid, amine or ester monomers, of urea-formaldehyde polymers or of vinylidene chloride polymers or copolymers; for example, microcapsules made of methyl acrylate or methacrylate polymers or copolymers, and alternatively of copolymers of vinylidene chloride and of acrylonitrile, for example, “Expancel®” from the company Expancel. These polymers include, for example, those having from 20 to 60% by weight of units derived from vinylidene chloride, from 20 to 60% by weight of units derived from acrylonitrile and from 0 to 40% by weight of other units such as units derived from an acrylic and/or styrene monomer; crosslinked acrylic polymers and copolymers may also be used,
elastomeric crosslinked organopolysiloxane powders, described for example, in document JP 2,243,612 A, such as those sold under the name “Trefil Powder E-506C” by the company Dow Corning, elastomeric crosslinked organopolysiloxane powders coated with silicone resin, such as silsesquioxane resin, as described, for example, in patent U.S. Pat. No. 5,538,793. Such elastomers are sold under the names “KSP-100”, “KSP-101”, “KSP-102”, “KSP-103”, “KSP-104” and “KSP-105” by the company Shin-Etsu, and mixtures thereof.

Low-Density Fillers

In other embodiments, the compositions comprise at least one low-density filler in an amount of at least 5% by weight relative to the total weight of the composition.

The term “low-density filler,” as used herein, means a filler whose untamped density, measured via the protocol given below, ranges from 0.01 to 0.50 g/cm³, for example, ranging from 0.05 to 0.50 g/cm³ or from 0.10 to 0.50 g/cm³.

Protocol for Measuring Untamped Density (D_{UT}):

A volume of from 240 to 250 ml of powder is poured into a 250 ml measuring cylinder having a mass M₀ (g), using a funnel. The volume V₀ (cm³) of powder poured in is then read on the cylinder and the cylinder filled with powder is weighed to measure the mass M₁ (g).

The untamped density is determined according to the following formula:
D_{UT}=(M₁−M₀)/V₀

The low-density fillers may also be characterized by a tamped density, measured via the protocol given below, ranging from 0.01 to 0.80 g/cm³, for example, ranging from 0.05 to 0.80 g/cm³ or from 0.10 to 0.80 g/cm³.

Protocol for Measuring Tamped Density (D_{T}):

A volume of from 240 to 250 ml of powder is poured into a 250 ml measuring cylinder having a mass M₀ (g) using a funnel. The volume V₀ (cm³) of powder poured in is then read on the cylinder and the cylinder filled with powder is weighed to measure the mass M₁ (g).

The cylinder is then placed on the STAV 2003@ machine from Stampf Volumeter. The cylinder is then subjected to a series of 2500 tamping blows, and the volume Vₙ (volume after the n^{th} series of 2500 tamping blows) of the powder in the cylinder is then measured at the end of each series.

Once Vₙ−Vₙ₊₁≦(2×Vₙ)/100, the tamping of the cylinder is stopped and the volume Vₙ (cm³) is noted.

The tamped density is determined according to the following relationship:
D_{T}=(M₁−M₀)/Vₙ

The low-density fillers useful herein may be chosen from the list of incompactable fillers given above as well as from the fillers described in the following table:

| | Untamped density | Untamped density |
|---|---|---|
| Low-density fillers | (g/cm³) | (g/cm³) |
| Sunsphere H-33 ® | 0.10 | 0.14 |
| from the company Asahi Glass | | |
| Trefil Powder E-506C ® | 0.19 | 0.27 |
| from the company Dow Corning | | |
| KSP-100 ® | 0.27 | 0.39 |
| from the company Shin-Etsu | | |
| Silica Beads SB 700 ® | 0.28 | 0.48 |
| from the company Maprecos | | |
| Sunsphere H-51 ® | 0.28 | 0.49 |
| from the company Asahi Glass | | |
| Micropearl M-100 ® | 0.39 | 0.64 |
| from the company SEPPIC | | |
| Plastic Powder D-400 ® | 0.46 | 0.76 |
| from the company Toshiki | | |
| Polytrap 6603 Adsorber ® | 0.05 to 0.12 | 0.07 to 0.10 |
| from the company RP Scherrer | | |

The composition according to the present disclosure may comprise at least one incompactable filler or a mixture of at least one incompactable filler and/or at least one low-density filler in an amount ranging from 5% to 15% by weight, for example from 5% to 13% or 5% to 10% by weight relative to the total weight of the composition.

The pulverulent phase may also further comprise at least one additional filler. These fillers may be mineral or organic and of any shape, such as platelet-shaped, spherical or oblong, irrespective of their crystallographic form (for example lamellar, cubic, hexagonal, orthorhombic, etc.). Additional fillers that may be used in the compositions include talc, mica, silica, kaolin, polyamide (Nylon®) powder, poly-β-alanine powder, polyethylene powder, tetrafluoroethylene polymer (Teflon®) powders, lauroyllysine, starch, boron nitride, acrylic acid polymer powders, silicone resin microbeads (for example Tospearls® from Toshiba), precipitated calcium carbonate, magnesium carbonate, magnesium hydrocarbonate, hydroxyapatite, hollow silica microspheres, and glass or ceramic microcapsules.

The additional filler or mixture of additional fillers may be present in an amount ranging from 40% to 95% by weight, for example, from 45% to 85% or 45% to 70% by weight relative to the total weight of the composition.

As mentioned above, the compositions according to the present disclosure may also comprise at least one pulverulent dyestuff chosen from pigments and nacres.

The pigments may be white or colored, mineral and/or organic, and coated or uncoated. Mineral pigments that may be used herein include titanium dioxide, optionally surface-treated, zirconium oxide or cerium oxide, and also iron oxide or chromium oxide, manganese violet, ultramarine blue, chromium hydrate and ferric blue. Organic pigments that may be used include carbon black, pigments of D & C type and lakes based on cochineal carmine or on barium, strontium, calcium or aluminium.

The nacres may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type and nacreous pigments based on bismuth oxychloride.

The pulverulent dyestuff may be present in the compositions in an amount ranging from 0.5% to 30% by weight, for example from 1% to 22% or 3% to 18% by weight relative to the total weight of the composition.

Fatty Phase:

As mentioned above, the fatty phase may also be referred to as a “binder” and serves as a dispersing medium for the particulate phase. The fatty phase may be present in the presently disclosed compositions in an amount ranging from 12% to 35% by weight, for example, from 15% to 30% by weight relative to the total weight of the composition.

Solid Fatty Phase:

The compositions as disclosed herein comprise at least one fatty phase comprising at least one solid fatty phase, also known as a “solid binder” in an amount such that the weight ratio of the at least one solid fatty phase to the at least one incompactable filler and/or at least one low-density filler is greater than or equal to 1, such as greater than or equal to 1.5.

As used herein, the term “solid binder” is understood to mean a fatty phase whose melting point may be greater than or equal to 30° C., for instance ranging from 30° C. to 250° C., such as from 30° C. to 230° C.

The at least one solid fatty phase may comprise at least one compound chosen from waxes and metal soaps, and mixtures thereof.

As used herein, the term “wax” is understood to mean a lipophilic fatty compound, which is solid at room temperature and atmospheric pressure (760 mmHg, i.e. 10⁵ Pa), which undergoes a reversible solid/liquid change of state, and which for instance has a melting point of greater than or equal to 30° C., for example, greater than or equal to 55° C., such as up to 120° C., which may be up to 250° C., such as up to 230° C.

By bringing the wax to its melting point, it is possible to make it miscible with the oils and to form a microscopically homogeneous mixture, but upon returning the temperature of the mixture to room temperature, the recrystallization of the wax in the oils of the mixture is obtained.

According to the present disclosure, the melting point values correspond to the melting peak measured using a differential scanning calorimeter (DSC), for example the calorimeter sold under the name DSC 30 by the company Mettler, with a temperature rise of 5 or 10° C. per minute.

For the purposes of the present disclosure, the waxes may be those generally used in cosmetics or dermatology. They may be, for example, hydrocarbon-based waxes, silicone waxes and/or fluoro waxes, optionally comprising ester or hydroxyl functional groups. They may also be of natural or synthetic origin.

Non-limiting illustrations of the waxes that may be used may be made of:
beeswax, lanolin wax and Chinese insect waxes; rice wax, carnauba wax, candelilla wax, ouricury wax, cork fiber wax, sugarcane wax, Japan wax and sumach wax; montan wax; microcrystalline waxes, paraffin waxes, ozokerites, ceresin wax, lignite waxes, polyethylene waxes, the waxes obtained by Fisher-Tropsch synthesis, and fatty acid esters and glycerides that are solid at 40° C. and above, for example, above 55° C.,
the waxes obtained by catalytic hydrogenation of animal or plant oils comprising linear and branched C₈-C₃₂ fatty chains, such as hydrogenated jojoba oil, hydrogenated sunflower oil, hydrogenated castor oil, hydrogenated coconut oil and hydrogenated lanolin oil,
silicone waxes or fluoro waxes, and
mixtures thereof.

According to one embodiment of the present disclosure, the at least one solid fatty phase may comprise at least one wax chosen from carnauba waxes and paraffin waxes, and mixtures thereof.

According to another embodiment of the present disclosure, the at least one solid fatty phase may comprise at least one wax that is totally or partially in powder form, such as in micronized form, to facilitate its use in the preparation of the cosmetic composition.

Among the waxes that may be used in powder form, non-limiting mention may be made of, for example, the carnauba wax microbeads sold under the name Microcare 350® by the company Micro Powders and the paraffin wax microbeads sold under the name Microease 114S® by the company Micro Powders.

The solid binder may also be chosen from metal soaps.

Among these soaps, non-limiting mention may be made of, for example, the metal soaps of fatty acids comprising from 12 to 22 carbon atoms, such as those comprising from 12 to 18 carbon atoms.

In one embodiment of the present disclosure, the metal of the metal soap may be chosen from zinc and magnesium.

The fatty acid may be chosen from, for example, lauric acid, myristic acid, stearic acid and palmitic acid.

Non-limiting examples of metal soaps that may be used include zinc laurate, magnesium stearate, magnesium myristate and zinc stearate, and mixtures thereof.

According to one aspect of the present disclosure, the at least one solid fatty phase may comprise at least one metal soap that is totally or partially present in powder form.

The at least one solid fatty phase may be present in an amount greater than or equal to 35% by weight, for instance greater than or equal to 40% by weight, such as greater than or equal to 50% by weight, relative to the total weight of the fatty phase.

The weight ratio of the at least one solid fatty phase to the at least one incompactable filler or at least one low-density filler can range, for example, from 1 to 4, for instance, from 1 to 3, such as from 1 to 2.5.

The composition as disclosed herein can comprise the at least one solid fatty phase, for example, in an amount ranging from 8% to 25% by weight, such as from 10% to 22% by weight, and from 10% to 20% by weight relative to the total weight of the composition.

Liquid Fatty Phase:

The fatty phase of the composition according to the present disclosure may also comprise a liquid fatty phase comprising at least one oil. This oil may be chosen from the oils conventionally used as binders in compact powders.

Non-limiting examples of the oils that may be used include:
mink oil, turtle oil, soybean oil, grapeseed oil, sesame seed oil, corn oil, rapeseed oil, sunflower oil, cotton seed oil, avocado oil, olive oil, castor oil, jojoba oil and groundnut oil;
hydrocarbon oils such as liquid paraffins, squalane, petroleum jelly, and polydecene, for example, hydrogenated polydecene, for instance “Ceraflow E®” sold by the company Shamrock;
fatty esters, such as isopropyl myristate, isopropyl palmitate, butyl stearate, isodecyl stearate, hexyl laurate, isononyl isononanoate, 2-ethylhexyl palmitate, 2-hexyldecyl laurate, 2-octyidecyl palmitate, 2-octyldodecyl myristate or lactate, 2-diethylhexyl succinate, diisostearyl malate, and glyceryl or diglyceryl triisostearate;
silicone oils such as polymethylsiloxanes, polymethylphenylsiloxanes, polysiloxanes modified with fatty acids, fatty alcohols or polyoxyalkylenes, fluoro silicones or perfluoro oils;
higher fatty acids such as myristic acid, palmitic acid, stearic acid, behenic acid, oleic acid, linoleic acid, linolenic acid or isostearic acid;
higher fatty alcohols such as cetanol, stearyl alcohol or oleyl alcohol;
the polymethylfluoroalkyldimethylsiloxanes of formula (I): 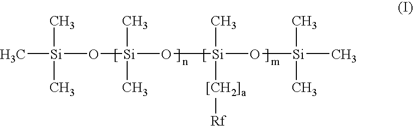
   wherein:
n is an integer ranging from 5 to 90, for instance from 30 to 80, such as from 50 to 80,
m is an integer ranging from 1 to 150, such as from 1 to 80, for instance from 1 to 40;
a is an integer ranging from 0 to 5, and
Rf is chosen from perfluoroalkyl radicals comprising from 1 to 8 carbon atoms; and
mixtures thereof.

Among the compounds of formula (I) that may be used as disclosed herein, non-limiting mention may be made of, for example, the products sold under the names X22-819®, X22-820®, X22-821® and X22-822® by the company Shin-Etsu.

The composition according to the present disclosure may also comprise a liquid fatty phase in an amount ranging from 4% to 15% by weight, for instance, from 6% to 13% by weight, such as from 7% to 13% by weight, relative to the total weight of the composition.

According to one aspect of the present disclosure, the liquid fatty phase and the at least one incompactable filler or at least one low-density filler are present in the compositions as disclosed herein such that the weight ratio of the liquid fatty phase to the at least one incompactable filler or at least one low-density filler is less than or equal to 3, such as than or equal to 2.5, for instance less than or equal to 2 or even less than or equal to 1.5.

According to one embodiment of the present disclosure, the composition is free of liquid fatty phase.

Needless to say, a person skilled in the art will take care to adjust the amounts of the at least one solid fatty phase and possibly the liquid phase of the composition according to the present disclosure such that the expected properties in terms of cohesion and matting effect over time are satisfactory.

Additives:

The composition may also comprise at least one adjuvant often used in cosmetics, such as water-soluble or liposoluble coloring agents, preserving agents, cosmetic active agents, moisturizers, UV-screening agents, thickeners, water, surfactants and/or fragrances.

Needless to say, a person skilled in the art will take care to select the optional at least one adjuvant added to the compositions such that the beneficial properties intrinsically associated with the compositions are not, or are not substantially, adversely affected by the envisaged addition.

The compositions as disclosed herein can be in the form of a makeup product of compact powder type, such as a makeup rouge, an eyeshadow, a face powder, a foundation, a concealer product or a body makeup product, or alternatively in the form of a facial care product or a body care product.

The invention is illustrated in greater detail by the non-limiting examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE 1

### Reference Liquid Binder

| | |
|---|---|
| 2-ethylhexyl 4-para-methoxycinnamate | 40.91 g |
| (Parsol ® MCX from the company Roche Vitamins), | |
| isocetyl stearate | 25 g |
| polydimethylsiloxane 10 cst (Fluid DC 200 ® 10 cst | 25 g |
| from Dow Corning) and | |
| mixture of preserving agents Phenonip ® from the | 9.09 g |
| company Nipa (Clariant), described below | |

The mixture of preserving agents Phenonip® contains:

| | |
|---|---|
| 2-phenoxyethanol | 70 g |
| methyl p-hydroxybenzoate | 17.40 g |
| ethyl p-hydroxybenzoate | 4.20 g |
| butyl p-hydroxybenzoate | 4.20 g |
| propyl p-hydroxybenzoate, and | 2.10 g |
| isobutyl p-hydroxybenzoate | 2.10 g |

### EXAMPLE 2

### Foundation

(a) Composition: According to the Invention

| | |
|---|---|
| yellow, brown, black iron oxides | 3.23 g |
| amorphous silica microspheres: | 1.00 g |
| Sunsphere ® H-33 from Asahi-Glass | |
| preserving agents | qs |
| titanium dioxide | 10.55 g |
| sericite | 15.00 g |
| boron nitride | 6.00 g |
| talc | 28.30 g |
| polyurethane/silica powder (98/2): | 3.00 g |
| Plastic Powder ® D-400 from Toshiki | |
| titanium dioxide - zinc oxide - methylhydrogenopolysiloxane | 4.00 g |
| coated talc: TZ Powder ® type 2 from Myoshi | |
| crosslinked polydimethylsiloxane powder: | 2.00 g |
| Trefil Powder ® E-506 C from Dow Corning | |
| ethylene/acrylic acid copolymer powder: | 5.00 g |
| Flobeads ® EA-209 from Sumitomo | |
| paraffin wax microbeads: | 10.00 g |
| Microease ® 114S from Micro Powders | |
| magnesium stearate | 3.00 g |
| polymethyltrifluoropropyldimethylsiloxane: | 2.92 g |
| X22-819 ® from Shin-Etsu | |
| 2-ethylhexyl 4-p-methoxycinnamate | 4.00 g |
| isononyl isononanoate | 1.20 g |

The composition was prepared by mixing together the mixture of powders and then adding the fatty phase thereto, this mixture was then ground and screened until a homogeneous mixture was obtained. Some of the composition (10.5 g) was placed in a dish and then compacted at a pressure of 2×10⁷ Pa (200 bar).

The composition had a cohesion such that the loss of product during the drop test was 2% by weight.

Application of the compact powder to the face afforded a makeup result that showed good color homogeneity and good matting throughout the day.

### (b) COMPARATIVE EXAMPLE

91.2% by weight of the same pulverulent phase was formulated in the presence of 8.8% by weight of a liquid binder, i.e., 3.60 g of 2-ethylhexyl 4-p-methoxycinnamate, 2.20 g of polydimethylsiloxane and 2.20 g of isocetyl stearate and 0.80 g of a mixture of preserving agents:

| | |
|---|---|
| yellow, brown, black iron oxides | 3.77 g |
| amorphous silica microspheres: | 1.17 g |
| Sunsphere ® H-33 from Asahi-Glass | |
| preserving agents | qs |
| titanium dioxide | 12.32 g |
| sericite | 17.52 g |
| boron nitride | 7.01 g |
| talc | 33.06 g |
| polyurethane/silica powder (98/2): | 3.50 g |
| Plastic Powder ® D-400 from Toshiki | |
| titanium dioxide - zinc oxide - methylhydrogenopolysiloxane | 4.67 g |
| coated talc: TZ Powder ® type 2 from Myoshi | |
| crosslinked polydimethylsiloxane powder: | 2.34 g |
| Trefil Powder ® E-506 C from Dow Corning | |
| ethylene/acrylic acid copolymer powder: | 5.84 g |
| Flobeads ® EA-209 from Sumitomo | |
| 2-ethylhexyl 4-p-methoxycinnamate | 3.60 g |
| isocetyl stearate | 2.20 g |
| polydimethylsiloxane 10 cst (Fluid DC ® 200 10 cst from | 2.20 g |
| Dow Corning) | |
| mixture of preserving agents Phenonip ® from the company | 0.80 g |
| Nipa (Clariant) | |

The compacted composition was tested to evaluate its mechanical properties. The loss of product during the drop test was 17.6% by weight, which reflects a cohesion defect. In addition, the erosion of the compacted product with a dry sponge was found to be too great, since the product crumbled away upon contact with the sponge and the compact wore away by hollowing. The liquid binder therefore did not make it possible to obtain a satisfactory compact powder.

### EXAMPLE 3

### Compact Foundation:

(a) Composition According to the Invention

| | |
|---|---|
| yellow, brown, black iron oxides | 5.75 g |
| talc | 56.25 g |
| amorphous silica microspheres: Sunsphere ® H-33 | 10.00 g |
| from Asahi Glass | |
| carnauba wax microbeads: Microcare ® 350 from | 20.00 g |
| Micro Powders | |
| hydrogenated polydecene: Ceraflow E ® from Shamrock | 3.00 g |
| polymethyltrifluoropropyldimethylsiloxane: | 5.00 g |
| X22-819 ® from Shin-Etsu | |

The composition was prepared by mixing together the mixture of powders (iron oxides, talc, silica and carnauba wax) and then adding the oils (hydrogenated polydecene and fluorosilicone) thereto, and this mixture was then ground and screened until a homogeneous mixture was obtained. Some of the composition (10.5 g) was placed in a dish and then compacted at a pressure of 2×10⁷ Pa (200 bar).

The compacted composition had a cohesion such that the loss of product during the drop test was less than 5.72% by weight.

The compact powder obtained afforded a makeup result that showed good matting throughout the day when applied to the face.

(b) Comparative Composition 91.2% by weight of the same pulverulent phase was formulated in the presence of 8.8% by weight of a liquid binder, i.e., 3.60 g of 2-ethylhexyl 4-p-methoxycinnamate, 2.20 g of polydimethylsiloxane and 2.20 g of isocetyl stearate, and 0.80 g of a mixture of preserving agents:

| | |
|---|---|
| yellow, brown, black iron oxides | 7.27 g |
| talc | 71.26 g |
| amorphous silica microspheres: Sunsphere ® H-33 | 12.67 g |
| from Asahi Glass | |
| 2-ethylhexyl 4-p-methoxycinnamate | 3.60 g |
| isocetyl stearate | 2.20 g |
| polydimethylsiloxane 10 cst (Fluid DC ® 200 10 cst | 2.20 g |
| from Dow Corning) | |
| mixture of preserving agents Phenonip ® from | 0.80 g |
| the company Nipa (Clariant) | |

The composition was prepared in the same manner as described in (a).

The compacted composition had a cohesion such that the loss of product during the drop test was greater than 12% by weight. The erosion of the compacted product was too great. The product crumbled away on contact with a dry sponge and the compact was worn away by hollowing. The liquid binder therefore did not make it possible to obtain a compact powder that was satisfactory in terms of cohesion and erosion.

### EXAMPLE 3

### Foundation

(a) Composition According to the Invention:

| | |
|---|---|
| yellow, brown, black iron oxides | 3.23 g |
| lauroyllysine-coated titanium dioxide (95/5) | 9.00 g |
| methylhydrogenopolysiloxane-coated titanium dioxide-zinc | 4.00 g |
| oxide-talc: TZ Powder ® type 2 from Miyoshi | |
| preserving agents | qs |
| coated talc (SNI TA 68R ® from Miyoshi) | 30.80 g |
| amorphous silica microspheres: | 1.00 g |
| Sunsphere ® H-33 from Asahi Glass | |
| silica microbeads: | 1.00 g |
| Silica Beads ® SB150 from Miyoshi | |
| polyurethane/silica powder (98/2): | 5.00 g |
| Plastic Powder ® D-400 from Toshiki | |
| ethylene/acrylic acid copolymer powder: | 5.00 g |
| Flobeads ® EA-209 from Sumitomo | |
| nylon powder: | 5.00 g |
| Orgasol ® 2002 Extra D Nat Cos | |
| crosslinked polydimethylsiloxane powder: | 2.00 g |
| Trefil Powder ® E-506 C from Dow Corning | |
| paraffin wax microbeads: | 8.00 g |
| Microease ® 114S from Micro Powders | |
| sericite coated with carnauba wax (94.5/5.5): | 15.00 g |
| Sericite UW-A5 ® from Toshiki | |
| magnesium stearate | 3.00 g |
| isononyl isononanoate | 1.20 g |
| polymethyltrifluoropropyldimethylsiloxane: | 2.00 g |
| X22-819 ® from Shin-Etsu | |
| 2-ethylhexyl 4-p-methoxycinnamate | 4.00 g |

The composition was prepared by mixing together the mixture of powders (iron oxides, silica, polyurethane, etc.) and then adding the oils (isononyl isononanoate, fluorosilicone and 2-ethylhexyl 4-p-methoxycinnamate) thereto, and this mixture was then ground and screened until a homogeneous mixture was obtained. Some of this composition (10.5 g) was placed in a dish and then compacted at a pressure of 2×10⁷ Pa (200 bar).

The compacted composition had a cohesion such that the loss of product during the drop test was 2.2% by weight.

The compact powder foundation obtained afforded a makeup result that showed good matting throughout the day when applied to the face.

### b) COMPARATIVE EXAMPLE

91.2% by weight of the same pulverulent phase was formulated in the presence of 8.8% by weight of a liquid binder, i.e. 3.60 g of 2-ethylhexyl 4-p-methoxycinnamate, 2.20 g of polydimethyl siloxane and 2.20 g of isocetyl stearate and 0.80 g of a mixture of preserving agents:

| | |
|---|---|
| yellow, brown, black iron oxides | 3.64 g |
| lauroyllysine-coated titanium dioxide (95/5) | 10.13 g |
| methylhydrogenopolysiloxane-coated titanium dioxide-zinc | 4.50 g |
| oxide-talc: TZ Powder ® type 2 from Miyoshi | |
| preserving agents | qs |
| coated talc (SNI TA 68R ® from Miyoshi) | 34.65 g |
| amorphous silica microspheres: | 1.13 g |
| Sunsphere ® H-33 from Asahi Glass | |
| silica microbeads: | 1.12 g |
| Silica Beads ® SB150 from Miyoshi | |
| polyurethane/silica powder (98/2): | 5.63 g |
| Plastic Powder ® D-400 from Toshiki | |
| ethylene/acrylic acid copolymer powder: | 5.63 g |
| Flobeads ® EA-209 from Sumitomo | |
| nylon powder: | 5.63 g |
| Orgasol ® 2002 Extra D Nat Cos | |
| crosslinked polydimethylsiloxane powder: | 2.25 g |
| Trefil Powder ® E-506 C from Dow Corning | |
| sericite coated with carnauba wax (94.5/5.5): | 16.89 g |
| Sericite UW-A5 ® from Toshiki | |
| 2-ethylhexyl 4-p-methoxycinnamate | 3.60 g |
| isocetyl stearate | 2.20 g |
| polydimethylsiloxane 10 cst (Fluid DC ® 200 10 cst from | 2.20 g |
| Dow Corning) | |
| mixture of preserving agents Phenonip ® from the company | 0.80 g |
| Nipa (Clariant) | |

The compacted composition had a cohesion such that the loss of product during the drop test was 2.38% by weight.

However, the erosion of the compacted product was too great, the product crumbles away on contact with the sponge and the compact becomes worn away by hollowing. The liquid binder therefore did not make it possible to obtain a compact powder that shows satisfactory erosion.

Although the present disclosure has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present present disclosure as defined by the appended claims.

1. A cosmetic composition for making up and/or caring for the skin comprising: a pulverulent phase,
a solid fatty phase, and
a liquid fatty phase in an amount ranging from 4% to 15% by weight, relative to the total weight of the composition,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition, and
wherein the cosmetic composition is in compact powder form.

2. A cosmetic composition for making up and/or caring for the skin comprising:
a pulverulent phase, and
a fatty phase present in an amount ranging from 12% to 35% by weight, relative to the total weight of the composition, wherein the fatty phase comprises a solid fatty phase in an amount ranging from 8% to 25% by weight, relative to the total weight of the composition,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition, and
wherein the cosmetic composition is in compact powder form.

3. A cosmetic composition in the form of a compact powder for making up and/or caring for the skin comprising
a pulverulent phase, and
a fatty phase comprising at least one solid fatty phase,
wherein the at least one solid fatty phase is present in an amount such that the compact powder has a cohesion corresponding to a loss of mass, evaluated by a drop test, of less than 12% by weight, and
the pulverulent phase being such that when it is mixed in an amount of 91.2% by weight a with 8.8% of a liquid binder comprising 2-ethylhexyl 4-para-methoxycinnamate, isocetyl stearate, polydimethylsiloxane 10 cst, and a mixture of preserving agents, and when the whole is compacted at a pressure of 2×10⁷ Pa, it results in a compact whose cohesion corresponds to a loss of mass, evaluated by a drop test, of greater than 12% by weight and/or a compact that does not erode homogeneously,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition.

4. The composition according to claim 1, wherein the pulverulent phase comprises at least one filler chosen from incompactable fillers, low-density fillers, and pulverulent dyestuffs.

5. A cosmetic composition for making up and/or caring for the skin, comprising:
a pulverulent phase comprising at least one incompactable filler in an amount greater than or equal to 5% by weight, relative to the total weight of the composition, and
at least one fatty phase comprising at least one solid fatty phase,
the solid fatty phase being present in an amount such that the weight ratio of the at least one solid fatty phase to the at least one incompactable filler is greater than or equal to 1,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition, and
wherein the cosmetic composition is in compact powder form.

6. The composition according to claim 5, wherein the at least one incompactable filler is chosen from incompactable fillers of mineral type and organic type.

7. The composition according to claim 5, wherein that the at least one incompactable filler is chosen from silica microspheres, microporous polymer microspheres, polyurethane powder, polymer microcapsules bearing only one closed cavity, and elastomeric crosslinked organopolysiloxane powders.

8. The composition according to claim 7, wherein the at least one incompactable filler is chosen from silica microspheres of open porosity, hollow silica microspheres, microporous acrylic polymer microspheres, powdered copolymer of hexamethylene diisocyanate and of trimethylolhexyl lactone, microcapsules of methyl acrylate or methacrylate polymers or copolymers, copolymers of vinylidene chloride and of acrylonitrile, and powders of elastomeric crosslinked organopolysiloxane coated with silsesquioxane resin.

9. A cosmetic composition for making up and/or caring for the skin, comprising:
a pulverulent phase comprising at least one low-density filler having an untamped density ranging from 0.01 to 0.50 g/cm³ present in an amount greater than or equal to 5% by weight, relative to the total weight of the composition, and
at least one fatty phase comprising at least one solid fatty phase, the solid fatty phase being present in an amount such that the weight ratio of the at least one solid fatty phase to the at least one low-density filler is greater than or equal to 1,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition, and
wherein the cosmetic composition is in compact powder form.

10. The composition according to claim 9, wherein the at least one low-density filler has an untamped density ranging from 0.05 to 0.50 g/cm³.

11. The composition according to claim 9, wherein the at least one low-density filler has a tamped density ranging from 0.01 to 0.80 g/cm³.

12. The composition according to claim 9, wherein the at least one low-density filler is chosen from silica microspheres, microporous polymer microspheres, polyurethane powder, polymer microcapsules bearing only one closed cavity, and elastomeric crosslinked organopolysiloxane powders.

13. The composition according to claim 4, wherein the at least one filler is chosen from low density fillers and incompactable fillers and is present in an amount ranging from 5% to 15% by weight, relative to the total weight of the composition.

14. The composition according to claim 1, wherein the at least one solid fatty phase comprises at least one compound chosen from waxes and metal soaps.

15. The composition according to claim 1, wherein the solid fatty phase comprises at least one compound chosen from:
beeswax, lanolin wax, Chinese insect waxes, rice wax, carnauba wax, candelilla wax, ouricury wax, cork fiber wax, sugarcane wax, Japan wax, sumach wax, montan wax; microcrystalline waxes, paraffin waxes, ozokerites, ceresin wax, lignite waxes, polyethylene waxes, waxes obtained by Fisher-Tropsch synthesis, fatty acid esters of glycerides that are solid at 40° C.,
waxes obtained by catalytic hydrogenation of animal and plant oils comprising linear and branched C₈-C₃₂ fatty chains, hydrogenated sunflower oil, hydrogenated castor oil, hydrogenated coconut oil, hydrogenated lanolin oil, silicone waxes, fluoro waxes, zinc laurate, magnesium stearate, magnesium myristate, and zinc stearate.

16. The composition according to claim 1, wherein the solid fatty phase comprises at least one compound chosen from paraffin waxes and carnauba waxes.

17. The composition according to claim 1, wherein the solid fatty phase comprises at least one wax that is at least partially-in powder form.

18. The composition according to claim 1, wherein the solid fatty phase comprises at least one metal soap chosen from zinc laurate, magnesium stearate, magnesium myristate, and zinc stearate.

19. The composition according to claim 1, wherein the solid fatty phase is present in an amount of at least 35% by weight relative to the total weight of the fatty phase.

20. The composition according to claim 1, wherein the solid fatty phase is present in an amount ranging from 10% to 22% by weight, relative to the total weight of the composition.

21. The composition according to claim 2, wherein the weight ratio of the solid fatty phase to the pulverent phase ranges from 1 to 4.

22. The composition according to claim 1, wherein the fatty phase further comprises a liquid fatty phase comprising at least one oil chosen from: mink oil, turtle oil, soybean oil, grapeseed oil, sesame seed oil, corn oil, rapeseed oil, sunflower oil, cotton seed oil, avocado oil, olive oil, castor oil, jojoba oil, groundnut oil, hydrocarbon oils, fatty esters, silicone oils, higher fatty acids, higher fatty alcohols, and polymethylfluoroalkyldimethylsiloxanes of formula (I): 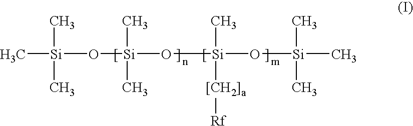
wherein:
n is an integer ranging from 5 to 90,
m is an integer ranging from 1 to 150,
a is an integer ranging from 0 to 5, and
Rf is chosen from perfluoroalkyl radicals comprising from 1 to 8 carbon atoms.

23. The composition according to claim 22, wherein the weight ratio of the liquid fatty phase to the pulverent phase is less than or equal to 3.

24. The composition according to claim 22, wherein the liquid fatty phase is present in an amount ranging from 6% to 13% by weight, relative to the total weight of the composition.

25. The composition according to claim 1, wherein the total content of fatty phase ranges from 15% to 30% by weight, relative to the total weight of the composition.

26. The composition according to claim 1, furthur comprising at least one additional filler.

27. The composition according to claim 26, wherein at least one additional filler is chosen from talc, mica, silica, kaolin, polyamide powder, poly-β-alanine powder, polyethylene powder, tetrafluoroethylene polymer powders, lauroyllysine, starch, boron nitride, acrylic acid polymer powders, silicone resin microbeads, precipitated calcium carbonate, magnesium carbonate, magnesium hydrocarbonate, hydroxyapatite, hollow silica microspheres, and glass or ceramic microcapsules.

28. The composition according to claim 26, wherein the at least one additional filler is present in an amount ranging from 40% to 95% by weight, relative to the total weight of the composition.

29. The composition according to claim 1, furthur comprising at least one pulverulent dyestuff.

30. The composition according to claim 29, wherein the at least one pulverulent dyestuff is chosen from pigments and nacres.

31. The composition according to claim 29, wherein the at least one pulverulent dyestuff is present in an amount ranging from 0.5% to 30% by weight, relative to the total weight of the composition.

32. The composition according to claim 1, furthur comprising at least one adjuvant chosen from liposoluble and water-soluble coloring agents, preserving agents, cosmetic active agents, moisturizers, UV-screening agents, thickeners, water, surfactants, and fragrances.

33. The composition according to claim 1, wherein the composition is in a form chosen from a makeup rouge, an eyeshadow, a face powder, a foundation, a concealer product, a body makeup product, a facial care product, and a body care product.

34. The composition according to claim 33, wherein the composition is in the form of a foundation.

35. A method of preparing a cosmetic composition in compact powder form, comprising mixing
a pulverulent phase, in an amount greater than or equal to 5% by weight, relative to the total weight of the composition, comprising at least one filler chosen from incompactable fillers and low-density fillers, and
at least one fatty phase comprising at least one solid fatty phase, wherein the at least one solid fatty phase is present in an amount such that the compact powder has a cohesion corresponding to a loss of mass, evaluated by a drop test, of less than 12% by weight, and
wherein the pulverulent phase is such that when it is mixed in an amount of 91.2% by weight a with 8.8% of a liquid binder comprising 2-ethylhexyl 4-para-methoxycinnamate, isocetyl stearate, polydimethylsiloxane 10 cst, and a mixture of preserving agents, and when the whole is compacted at a pressure of 2×10⁷ Pa, it results in a compact whose cohesion corresponds to a loss of mass, evaluated by a drop test, of greater than 12% by weight and/or a compact that does not erode homogeneously,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition, and
wherein the pulverulent phase and fatty phase are present in an amount effective to provide satisfactory cohesion properties.

36. A method for making up and/or caring for the skin, comprising applying to the skin at least one composition comprising:
a pulverulent phase,
a solid fatty phase, and
a liquid fatty phase present in an amount ranging from 4% to 15% by weight, relative to the total weight of the composition,
with the proviso that the cosmetic composition does not include a composition comprising 10% by weight of carnauba wax, 6% by weight of magnesium stearate and 5% by weight of amorphous magnesium and/or aluminium silicate relative to the total weight of the composition, and
wherein the composition is applied to the skin in an amount effective to convey a matte effect to the skin.
